(19) United States
(12) Patent Application Publication
(10) Pub. No.: US 2005/0075276 A1
(43) Pub. Date: Apr. 7, 2005

(54) Use of inhibitors of glycogen synthase-3 to augment CD28 dependent -T-cell responses

(76) Inventor: Christopher Rudd, Cambridge, MA (US)

Correspondence Address: Christopher Rudd;Immune Ventures Corp., 39E Bellis Circle, Cambridge, MA, 02140, US

(21) Appl. No.: 10/800,985

(22) Filed: Mar. 14, 2004

Related U.S. Application Data
(60) Provisional application No. 60/455,003, filed on Mar. 14, 2003.

(51) Int. Cl. A61K 38/52; A61K 31/44; A61K 31/505; A61K 31/405; A61K 33/14

(52) U.S. Cl. 514/2; 514/414; 514/256; 514/277; 424/677

(57) ABSTRACT

The invention concerns the identification of a pathway where the co-receptor CD28 inactivates glycogen synthase 3 (GSK-3) and where a reagent that inhibits GSK-3 can enhance, or substitute for CD28-dependent T-cell mediated immune responses. Altered T-cell responses are applied to increased proliferation, cytokine production and in the generation of increased cytolytic T-cell responses (CTLs) in the context of infection by virus', bacterial, fungi or prions. For example, the use of GSK-3 inhibitor SB415286 preferentially cooperated with CD28 costimulation to increase interleukin 2 (IL-2) transcription, and to provide CD28-replacement signals leading to increased IL-2 transcription. Similarly, inhibitors SB415286 and lithium chloride increased the CD28-dependent generation of cytolytic T-cell responses (CTLs) against a virally or baterially infected cells. Overall, the invention relates to a CD28 induced signaling transduction pathway that inactivates glycogen synthase kinase (GSK) and the application of this finding to the modulation of T-cell activation, proliferation and the generation of cytolytic T-cells (CTLs).

### FIELD OF INVENTION

The invention relates to treatment of infectious diseases and the identification of compounds useful in the treatment of infectious diseases and the development of vaccines.

The invention provides the use of drugs (non-peptides or peptides) that inactivate the kinase, glycogen synthase 3 (GSK-3α,β) to alter T-cell responses dependent on the co-receptor CD28. This involves the prevention of T-cell anergy induction, the production of cytokines by T-cells, the generation of cytolytic T-cell responses against virus', bacteria and other infectious agents and the development of transgenic nonhuman animals that comprise transgenes that alter CD28 inactivation of GSK-3α or β. The invention also provides methods for the identification of novel drugs or peptides useful in the treatment of infections that interfere or augment the CD28 inactivation of the GSK-3α,β pathway.

### BACKGROUND OF INVENTION

The immune response is coordinated by the activation of T-cells and their ability to proliferate, produce cytokines and development into effector T-cells. T-cells become activated by the engagement of the antigen-receptor (TcRζ/CD3) and co-receptors such as CD28, ICOS and others. Major histocompatibility complex (MHC) antigens on antigen-presenting cells (APCs) can process and present peptide antigen that is recognized by the TcRζ/CD3 complex. Nevertheless, while T-cells can be activated to proliferate in response to high avidity anti-CD3 antibody, the response of the majority of peripheral T-cell responses to antigenic peptide requires additional signals from the co-receptor CD28 for proliferation, cytokine production and effector function. The process of T-cell activation occurs over a period of 7-10 days and involves cell division, the production of cytokines needed for the generation of T-cell effector cells and B-cell responses as well as the differentiation of monocytes and granulocytes. T-cell activation also involves the induction of the activation of multiple genes requiring the production and activation of transcription factors such as NFkB, AP-1 and NFAT.

CD28 is a homodimeric member of the immunoglobulin supergene family that binds to the ligands CD80 and CD86 on antigen presenting cells (APCs). The dependency of peripheral T-cells responses on CD28 is demonstrated by a number of examples. For example, the ability of the mouse immune system to react and reject implanted tumor cells (i.e. melanoma) depends critically on ligation of CD28. In the absence of CD28 ligation, implanted tumors spread and kill the host animal (Chen et al Cell 1992 71:1093-102). Similarly, the response to most peptide antigens such as tetanus toxoid is dependent on CD28 ligation (Bluestone Immunity 2, 555-559 (1995); Alegre et al Nat. Rev. Immunol. 1, 220-228 (2001). CD28 is also needed for increased cytokine production (Bluestone, 1995 #66; Thompson, 1994 #1856], and for the prevention of anergy and apoptosis (Powell et al. Immunol. Rev. 165, 287-300 (1998); Schwartz Cell 71, 1065-1068 (1992)). It has also been implicated in Th1 vs. Th2 differentiation (Bluestone Immunity 2, 555-559 (1995)).

A key question has concerned the signaling events responsible for these CD28 mediated events (Rudd and Schneider 2003 Nat. Immunol. 3, 544-56). CD28 can bind to the lipid kinase phosphatidylinositol 3-kinase (PI 3K) and Grb-2 (Prasad et al. Proc. Natl. Acad. Sci. USA 91, 2834-2838 (1994); Schneider et al Eur. J. Immunol. 25, 1044-1050 (1995); Kim et al J. Biol. Chem. 273, 296-301 (1998); Yin et al J. Leukoc. Biol. 73, 178-182 (2003).). However, conflicting results have been obtained on whether the binding of these components is needed for CD28 co-stimulation (Cai et al Immunity 3, 417-426 (1995); Cefai et al Int. Immunol. 8, 1609-1616 (1996); Truitt et al. J. Immunol. 155, 4702-4710 (1995); Hutchcroft et al. Proc. Natl. Acad. Sci. USA 91, 3260-3264 (1995); Okkenhaug, et al. Nat Immunol 325, 325-332 (2001); Burr et al. J. Immunol. 166, 5331-5335 (2001); Harada et al. J. Immunol. 166, 3797-3803 (2001)).

In this context, the enzyme, glycogen synthase kinase (GSK) is a serine/threonine kinase that include two isoforms, alpha. and beta. Glycogen synthase kinase-3.beta. (GSK-3.beta.) was originally identified as a protein kinase that phosphorylated and inactivated glycogen synthase, an enzyme that regulates insulin-stimulated glycogen synthesis ((see Embi et al., Eur. J. Biochem. 107, 519-527, (1980); Rylatt et al., Eur. J. Biochem. 107, 529-537, (1980); and Vandenheede et al., J. Biol. Chem. 255, 11768-11774, (1980)). GSK-3.beta. is inhibited upon insulin activation, activating glycogen synthase. In this way, inhibition of GSK-3.beta. stimulates insulin-dependent processes and is useful in the treatment of type 2 diabetes. It has also been discovered that GSK-3.beta. plays a role in pathogenesis of Alzheimer's disease (see Lovestone et al., Current Biology, 4, 1077-86 (1994), Brownlees et al., Neuroreport, 8, 3251-3255 (1997), Takashima et al., PNAS 95, 9637-9641 (1998), and Pei et al., J Neuropathol. Exp., 56, 70-78 (1997)) and bipolar disorder (see Chen et al., J. Neurochemistry, 72, 1327-1330 (1999)).

In the immune system, GSK-3.beta. has been implicated in blocking of early immune response gene activation via nuclear factor of activated T-cells (NF-AT) (Beals et al., Science, 275, 1930-33 (1997); Pap, M. et al. J. Biochem. 273, 19929-19932, (1998)). In resting T-cells, NFAT exists in a phosphorylated state in the cytoplasm, while it is dephosphorylated upon TcR ligation by calcineurin (CaN), resulting in nuclear translocation and enhanced DNA-binding affinity. In the nucleus, phosphorylation by GSK-3 promotes NF-AT exit from the nucleus, thereby attenuating the function of the transcription factor. In T-cells, GSK-3 activity is decreased following T cell activation with PMA and ionomycin [Murphy, 2002]. Over-expression of a constitutively active form of GSK-3 in mouse T cells results in reduced IL-2 synthesis and proliferation. The duration of NFAT residency in the nucleus has been correlated with altered patterns of cytokine expression in T cells. GSK-3.beta is also required for the NF-kappa.B mediated survival response in the TNF-.alpha. signaling pathway.

This in turn is involved in the proinflammatory response to infection (Hoeflich et.al., Nature, 406, 86-90 (2000)). GSK-3.beta. is also known to regulate the degradation of a protein (beta.-catenin) which controls the activity of TCF family of transcription factors ((see., Dale, T. C., Biochem. J. 329, 209-223 (1998); Clevers, H. & van de Wetering, M., Trends in Genetics 13, 485-489 (1997); Staal, F. J. T. et al., International Immunology 11, 317-323 (1999)). GSK-3 also phosphorylates and regulates the function of c-Jun, c-Myc and eIF-2B (Doble and Woodgett, 2003 J Cell Sci. 116,1175-86). By phosphorylating c-jun and c-myc, GSK-3 regulates the ability of these transcription factors to control the expression of genes, while by phosphorylating eIF-2B, GSK-3 can regulate the translation of proteins needed for cell metabolism and function 9 (Doble and Woodgett, 2003 J Cell Sci. 116,1175-86). Our finding that the co-receptor CD28 phosphorylates and inactivates GSK-3 in T-cells provides a pathway that connects CD28 to the regulation of a variety of intracellular events in T-cells.

### SUMMARY OF THE INVENTION

The present invention provides several methods and compositions for modulating the immune response in relation to infection and infectious diseases and for screening for modulators of the immune response in relation to infectious diseases. These methods use drugs (peptides and non-peptides) that modulate the CD28 inactivation of GSK3α,β, or provide substitute signals leading to inactivation of GSK-3 in T-cells. Said drugs include lithium chloride and SB415286 from GlaxoSmithKline. The present invention also provides a method for identifying an agent useful in the treatment of infection, in which an agent that modulates CD28 inactivation of GSK-3α,β is identified The method can be carried out in a cell based assay employing T-cell lines or peripheral T-cells in conjunction with CD28 ligation and detection of phosphorylated GSK-3α on Ser-21 or GSK-3 β on Ser-9. Our invention outlines a signaling pathway by which CD28 co-signaling can occur in the enhancement of T-cell responses against foreign antigen, and provides for the use of an inhibitor of GSK-3 as a means of providing CD28-substitute or additive signals in the enhancement of T-cell immunity against virus', bacteria, prions and other infectious agents.

The first aspect of the invention provides a method of enhancing CD28 mediated and dependent T-cell responses against infectious agents in a mammalian subject comprising administering a drug to said subject, wherein said drug enhances or prolongs GSK-3α or β inactivation.

The method is considered to enhance the development of an immune response and the generation of a cytotoxic T-cell response (CTL) in the treatment of patients infected with virus', bacteria, prions and other infectious agents.

Infectious diseases in which the method of the invention may be particularly beneficial are those characterized by an increase in proliferation of T-cells, an increase in production of cytokines such as interleukin 2 and other interluekins as enhanced beyond that induced by TcR ligation, a reversal of apoptosis or cells death mediated by CD28 ligation and a reversal of anergy induction induced by TcR ligation.

Representative examples of infectious diseases dependent on T-cell responses are indicated below. Other examples of infection such as cytomegalovirus and others will be apparent to the skilled person.

TABLE 1
Infectious Agents

| Infectious Agents | Disease |
|---|---|
| Poliovirus | Polio |
| Measles virus | Measles |
| Mumps virus | Mumps |
| Rubella | German measles |
| Varicella zoster | Chickenpox |
| Influenza A | Influenza |
| Influenza B | Influenza |
| Rhinovirus | Upper respiratory infections |
| Adenovirus | Repiratory tract, enteric infections |
| Hepatitis A | Acute hepatitis A |
| Hepatitis B | Acute and chronic hepatitis B, cirrhosis, |
| | hepatoma |
| Hepatitis C | Acute and chronic hepatitis C, cirrhosis, |
| | hepatoma |
| Human | HIV related diseases |
| immunodeficiency virus | |
| Variola virus | Smallpox |
| Prions | Creutzfelt Jakob disease, Gerstmann-Straussler- |
| | Scheinker syndrome, Bovine spongiform |
| | encephalpathy |
| Corynebacterium | Diphtheria |
| diphtheriae | |
| Clostrium Tetani | Tetanus |
| Bordetella Pertussis | Pertussis |
| Pneumococcus | Meningitis, pneumonia, bacteremia, otitis |
| | media |
| Haemophilus Influenzae | Respiratory infections, meningitis |
| Vibrio Cholerae | Cholerae |
| Helicobacter Pylori | Gastritis, peptic ulcer disease, gastric |
| | neoplasms |
| Salmonellae | Typhoid fever, |
| Shigellae species | Shigellosis, gastroenteritis |
| Escherichia coli | Diarrhea, hemolytic uremic syndrome, urinary |
| | tract |
| Enterotoxigenic E. coli | infection |
| Enteropathogenic E. coli | |
| Enterohemorragic E. coli | |
| Enteroaggregative E. coli | |
| Plasmodia | Malaria |
| Other E. coli strains | |
| Schistosoma | Schistosomiasis |
| hematobium, S. mansoni, | |
| S. japonicum, | |
| S. mekongi, | |
| S. intercalatum | |
| Echinococcus | Echinococcal disease |

In another aspect, the invention provides a medicament for enhancing CD28 mediated and dependent T-cell responses against infectious agents in a mammalian subject, wherein the medicament comprises a drug which enhances or prolongs GSK-3α or β inactivation. The infectious agents includes virus', bacteria, prions and other infectious agents (see Table 1).

As a thrid aspect the invention provides a medicament for enhancing CD28 mediated and dependent T-cell responses against virus', bacteria, fungi and other infectious agents (examples in Table 1) in a mammalian subject wherein the medicament comprises a drug and is for administering to isolated peripheral T-cells or blood of said subject, wherein said drug enhances or prolongs GSK-3α or β inactivation, for example CD28-mediated GSK-3α or β inactivation. Cells may be re-infused into the patient at various cell concentrations, for example after being left for various times at 37° C. or RT.

In relation to each of the preceding aspects of the invention the mammalian subject may be a human. Alternatively, the recipient may be an animal, for example a domesticated animal (for example a dog or cat), laboratory animal (for example laboratory rodent, for example mouse, rat or rabbit) or animal important in agriculture (ie livestock), for example cattle, sheep or goats.

The method or medicament may also be useful in treating an immunocompromised state, for example arising from an infectious agent or other causes. Thus, the method or medicament may be useful in treating a patient who has been infected by virus', bacteria, fungi or prions. The method or medicament may be useful in regenerating and enhancing an immune response involving the production of cytokines (interleukin 2,4, interferony etc), the development of cytotoxic T-cells, and the reconstitution of immune cells in defense against infection.

The method or medicament may be useful for reversing the development of non-responsiveness or anergy to an infectious agent Thus, the method or medicament may be useful for treating a patient with an infection in which anergy appears to have been induced This can occurred as a result of high dose exposure to antigens from the infectious agent and as a result of ligation of the antigen receptor in the absence of CD28 expression or ligation (for example, signal one without signal two). This may be determined by assessing the response of the patient's immune cells to a defined antigen, peptide or in response to in vitro exposure to cells infected with the given virus' or bacterium Anergy is measured by the absence of a proliferative response, or production of cytokines such as interleukin 2, or alterations in the ability of TcR ligation to induce biochemical changes in the T-cell. Examples include alterations in the tyrosine phosphorylation of proteins or the induction of components such as cyclins that are needed for cell cycle progression. Anergy is also manifest in the inability of T-cells to migrate to lymph-nodes where they would encounter antigen provided by the infectious agent. Each of these events can be reversed by ligation of CD28. By substituting, complementing or cooperating with CD28 signaling, the method or medicament may be useful for reversing the development of non-responsiveness or anergy to an infectious agent.

The drug in relation to any of the preceding aspects of the invention may be any drug considered to inhibit GSK-3. The drug may be a peptide or a non-peptide drug. The drug may inhibit GSK-3α or GSK-3β or both. The drug may be SB415286 from GlaxoSmithKline, or a related GSK-3 inhibitory compounds such as a 3-indolyl-4-phenyl-1H-pyrrole-2,5-dione derivative, or other pyridine or pyrimidine derivative from other companies. The drug may be lithium chloride or a related compound.

Examples of GSK-3 inhibitors will be known to those skilled in the art. Examples are described in, for example, WO99/65897 and WO 03/074072 and references cited therein. For example, various GSK3 inhibitory compounds and methods of their synthesis and use are disclosed in U.S. and international patent application Publication Nos. 20020156087, WO0220495 and WO9965897 (pyrimidine and pyridine based compounds); 20030008866,20010044436 and WO0144246 (bicyclic based compounds); 20010034051 (pyrazine based compounds); and WO9816528 (purine based compounds). Further GSK3 inhibitory compounds include those disclosed in WO0222598 (quinolinone based compounds).

Further GSK-3 inhibitory compounds include macrocyclic maleimide selective GSK3β inhibitors developed by Johnson & Johnson and described in, for example, Kuo et al (Sep. 11, 2003) J Med Chem; 46(19):4021-31 Synthesis and discovery of macrocyclic polyoxygenated bis-7-azaindolylmaleimides as a novel series of potent and highly selective glycogen synthase kinase-3beta inhibitors. The bis-7-azaindolylmaleimides 28 and 29 are reported as exhibiting little or no inhibitions to a panel of 50 protein kinases. Compound 29 almost behaved as a GSK-3beta specific inhibitor. Both 28 and 29 displayed good potency in GS cell-based assay. A particular example is 10,11,13,14,16,17,19,20,22,23-Decahydro-9,4:24,29-dimetho-1H-dipyrido (2,3-n:3′,2′-t) pyrrolo (3,4-q)-(1,4,7,10,13,22) tetraoxadiazacyclotetracosine-1,3(2H)-dione.

The Pharmaprojects database indicates further GSK-3 inhibitors being developed by the following companies: Cyclacel (UK), Xcellsyz (UK)—XD-4241, Vertex Pharmaceuticals (USA)—eg VX-608, Chiron (USA), eg CHIR-73911, Kinetek (Canada) eg KP-354.

The documents indicated above relating to GSK3 inhibitors are hereby specifically incorporated by reference.

The drug can provide a CD28-substitute signal, or alternatively can act to cooperate in conjunction with reagents (i.e. anti-CD28 antibody; CD80/CD86) that ligate or crosslink the CD28 receptor. Anti-CD28 antibody may be provided as an intact antibody of any IG subclass, or as a part of an antibody such as a F(ab)2 or F(ab′) fragment. CD80 or CD86 or any newly discovered ligands for CD28 may be provided on cells, as complexes on other carriers such as yeast or as a soluble fusion protein (for example, CD80 fused to Ig). Thus, a method of the invention may further comprise the step of administering to the subject, or to the isolated peripheral T-cells or blood of the subject, a reagent (for example anti-CD28; anti-CD3/CD28; CD80/CD86) that ligates CD28. As well known to those skilled in the art the anti-CD28 may ligate (crosslink) CD28 by being immobilized on a surface or by being crosslinked, for example by a second reagent that binds to the anti-CD28, for example an anti-antibody antibody, for example as mentioned in the legends to FIGS. 1 and 2.

Similarly, in relation to the second and fourth aspects of the invention the mammalian subject, or the isolated peripheral T-cells or blood of the subject, is administered a reagent (for example anti-CD28; CD80/CD86) that ligates CD28. The reagent that ligates CD28 may be administered to the subject, or isolated peripheral T-cells or blood, before, after or simultaneously with the GSK3 inhibitor.

A further aspect of the invention provides a medicament comprising a reagent (for example anti-CD28; CD80/CD86) that ligates CD28 for enhancing CD28 mediated and dependent T-cell responses against infectious agents (examples in Table 1) in a mammalian subject, wherein the subject is a subject administered a non-peptide drug (either directly or via administration of the drug to isolated peripheral T-cells or blood of said subject) which enhances or prolongs GSK-3α or β inactivation, for example CD28-mediated GSK-3α or β inactivation.

A still further aspect of the invention provides a medicament comprising a reagent (for example anti-CD28; CD80/CD86) that ligates CD28 for enhancing CD28 mediated and dependent T-cell responses against infectious agents (examples in Table 1) in a mammalian subject, wherein the medicament is for administering to isolated peripheral T-cells or blood of said subject and wherein a drug which enhances or prolongs GSK-3α or β inactivation, for example CD28-mediated GSK-3α or β inactivation is administered to the isolated peripheral T-cells or blood of said subject or directly to the subject Cells may be re-infused into the patient at various cell concentrations, for example after being left for various times at 37° C. or RT.

The method of the invention (or use of the medicament) may lead to enhanced cytokine production and/or to increased T-cell proliferation and clonal expansion. The method of the invention (or use of the medicament) may alternatively or in addition lead to increased cytotoxic T-cell expansion and function.

A further aspect of the invention provides a method for expanding lymphoid cells that can be grown from an infected tissues, tissue-infiltrating lymphocytes, the method comprising administering a drug to material from the tissues from a mammalian subject, wherein said drug enhances or prolongs GSK-3α or β inactivation, for example CD28-mediated GSK-3α or β inactivation. Other examples include Hepatitis C infected liver peripheral blood cells infected with Human Immunodeficiency virus 1 (HIV-1) and others. The cells may in addition be administered a reagent that ligates CD28, as discussed above. The cells may re-infused into the subject at various cell concentrations (for example after being left for various times at 37° C. or RT) in order to enhance CD28 mediated and dependent T-cell responses against infectious agents in the subject.

A further aspect of the invention provides a medicament for treating infections, for example a infections as indicated above, comprising a composition or kit of parts comprising a GSK3 inhibitor and a reagent that ligates CD28. The composition and components of the kit of parts are preferably in a pharmaceutically acceptable form and may comprise a pharmaceutically acceptable carrier as discussed further below.

A further aspect of the invention provides a method for screening or identifying inhibitory or stimulatory compounds for use in treating infections (examples in Table 1), wherein the compounds are capable of modulating CD28 inactivation of GSK-3, the method comprising the step of exposing a T cell (either a peripheral T cell or T cell from a T cell line) that expresses CD28 and GSK3 to a test compound. The method may comprise the step of assessing phosphorylation of GSK3α on Ser 21 or GSK3β on Ser 9. Effects of the compound on other aspects of T cell behaviour, for example as investigated in the Figures and Examples, for example effect on ability to kill virally infected cells may also or alternatively be assessed.

In another embodiment, the invention provides a method for assessing the sensitivity of a subject to an immunosuppressive agent, for example, rapamycin or cyclosporin A. This relates to the instance where the immunosuppressive agent has a direct effect on CD28 induced function and signaling, or in the case where the agent suppresses signaling and function of another receptor, for example, TcR or CD2. The lymphocytes are obtained from a subject, incubated in the presence of the immunosuppressive agent at various doses and various times of incubation followed by a measurement of proliferation or cytokine production. This will be compared to lymphocytes that have not been exposed to the agent. Cells will be left as resting cells, or activated in vitro by anti-CD3 or anti-CD28 or anti-CD3/CD28, either as soluble antibody or immobilized on plates or on beads, for example, Dyna beads. In the case of a patient receiving immunosuppressive therapy, the response of lymphocytes are obtained from a subject will be compared to various normal patients who have not received the immuntherapy or to Cryo-perserved cells that were obtained from the patient prior to the onset of immunotherapy.

A further aspect of the invention provides a method of screening mammalian subjects infected with virus', bacteria and prions (examples in Table 1) for defects in the function of CD28 in providing co-stimulation for T-cells. CD28 (when ligated by antibody or natural ligand CD80/86) provides signals that augment cytokine production, increase energy metabolism, promote rescue from cells death (apoptosis), increase CTL generation, or promote rescue from anergy induction. This is the normal function for the co-receptor. In certain instances or in certain patients, CD28 may not function normally. This may be characterized by a loss of increased proliferation or production of cytokines such as interleukin 2, an inability to reverse apoptosis or cell death mediated by CD28 ligation and inability to reverse anergy induction induced by TcR ligation. The inability of CD28 to induce GSK-3 phosphorylation may be used as a screen to identify a defect in CD28 function. Alternatively or in addition it can be tested whether a drug that inhibits GSK-3 may mimic normal CD28 function under conditions where CD28 function itself is compromised. Use of the drug as a substitute for an aspect of CD28 function may provide for a screen for demonstrating that the upstream regulator of GSK-3 phosphorylation (i.e. CD28) is defective, despite perfectly normal GSK-3 function. The method may involve determining whether T cells derived from the subject show a response to a GSK3 inhibitor (for example phosphorylation of GSK3) that mimics the normal CD28 response, but do not show that response (for example phosphorylation of GSK3) when stimulated by CD28 ligation; or the response (for example GSK3 phosphorylation) is not inhibited by anti CD28 antibody.

The aforementioned therapeutic molecules, for example GSK3 inhibitor or reagent that ligates CD28, or a formulation thereof, may be administered by any conventional method including oral and parenteral (eg subcutaneous or intramuscular) injection. Preferred routes include oral, intranasal or intramuscular injection. Routes already known for GSK3 inhibitors may be used, though it will be appreciated that different localized treatment routes may be appropriate in the present invention than may be appropriate when treating (for example) diabetes. The treatment may consist of a single dose or a plurality of doses over a period of time.

As noted above, an adoptive therapy protocol may be used. Adoptive therapy protocols are described in Nestle et al (1998) Nature Med. 4, 328-332 and De Bruijn et al (1998) Cancer Res. 58, 724-731.

The therapeutic agent(s) may be given to a subject who is being treated for a disease that involves suppression of the immune response by some other method. This other mode of immunosuppression may act specifically to prevent the reactivity of a subset of cells to a specific antigen or infectious agent. However, given the non-specific nature of inhibition by the drug, for example cyclosporin A, it may be desirable to provide a second signal to enhance the function of another subset of cell by the CD28 receptor and its signaling pathway. For example, CD28 differentially regulates the development of T-helper 1 (Th1) and T-helper 2 (Th2) cells. The CD28 pathway and an inhibitor of GSK3 may overcome immunosuppression induced by these reagents, and activate the appearance of the desired subpopulation of T-cells. Thus, although the method of treatment may be used alone it is desirable to use it as an adjuvant therapy, for example alongside conventional preventative, therapeutic or palliative methods.

The therapeutic agent(s) may be given to a subject where the infection or a disease involves an abnormally low T-cell activation, or naturally immunosuppresses the subject. For example, infection by HIV-1 or HTLV-1 leads to a loss of reactive T-cells and the suppression of a response of intact T-cells. Induction of the CD28 pathway may be used to over-come, reverse or inhibit the suppression induced by these naturally infectious agents. One example is in the induction of cell death (apoptosis), or anergy where CD28 ligation has been shown to prevent this event. An inhibitor of GSK3 alone as a substitute signal, or in conjunction with CD28 ligation may provide a means to enhance the immune response against the infectious agent.

While it is possible for a therapeutic molecule as described herein, to be administered alone as an intramuscular or intravenous injection, or orally, as capsules, cachets or tablets, it is preferable to present it as a pharmaceutical formulation, together with one or more acceptable carriers. The carrier(s) must be “acceptable” in the sense of being compatible with the therapeutic molecule and not deleterious to the recipients thereof. Typically, the carriers will be water or saline which will be sterile and pyrogen free.

The formulations may conveniently be presented in unit dosage form and may be prepared by any of the methods well known in the art of pharmacy. Such methods include the step of bringing into association the active ingredient (for an antigenic molecule, construct or chimeric polypeptide of the invention) with the carrier that constitutes one or more accessory ingredients. The formulations are prepared by uniformly and intimately bringing into association the active ingredient with liquid carriers or finely divided solid carriers or both, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. Alternatively, the formulation may be administed to the patient by injection (examples, intramuscular, IV).

A tablet may be made by compression or moulding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured base, usually sucrose and acacia or tragacanth; pastilles comprising the active ingredient in an inert base such as gelatin and glycerin, or sucrose and acacia; and mouth-washes comprising the active ingredient in a suitable liquid carrier.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient.

It should be understood that in addition to the ingredients particularly mentioned above, the formulations of this invention may include other agents conventional in the art having regard to the type of formulation in question, for example those suitable for oral administration may include flavoring agents.

It will be appreciated that the therapeutic molecule can be delivered to the locus (for example the site of an infection) by any means appropriate for localised administration of a drug. For example, a solution of the therapeutic molecule can be injected directly to the site or can be delivered by infusion using an infusion pump. The therapeutic molecule also can be incorporated into an implantable device which when placed at the desired site, permits the therapeutic molecule to be released into the surrounding locus.

The therapeutic molecule may be administered via a hydrogel material. The hydrogel is non-inflammatory and biodegradable. Many such materials now are known, including those made from natural and synthetic polymers. In a preferred embodiment, the method exploits a hydrogel which is liquid below body temperature but gels to form a shape-retaining semisolid hydrogel at or near body temperature. Preferred hydrogel are polymers of ethylene oxide-propylene oxide repeating units. The properties of the polymer are dependent on the molecular weight of the polymer and the relative percentage of polyethylene oxide and polypropylene oxide in the polymer. Preferred hydrogels contain from about 10% to about 80% by weight ethylene oxide and from about 20% to about 90% by weight propylene oxide. A particularly preferred hydrogel contains about 70% polyethylene oxide and 30% polypropylene oxide. Hydrogels which can be used are available, for example, from BASF Corp., Parsippany, N.J., under the tradename Pluronic^{R}.

The molecule may be targeted to the required site (for example a site of infection or a site of antigen response, for example, the lymph-nodes and spleen) using a targeting moiety which binds to or lodges at the required site. A combined targeting/prodrug approach may be useful. When the targeting moiety is an antibody this type of system is often referred to as ADEPT (Antibody-Directed Enzyme Prodrug Therapy). The system requires that the targeting moiety locates the enzymatic portion to the desired site in the body of the patient (ie the site of infection) and after allowing time for the enzyme to localize at the site, administering a prodrug which is a substrate for the enzyme, the end product of the catalysis being the GSK3 inhibitor or CD28 or CD28/CD3 ligater.

The object of the approach is to maximise the concentration of drug at the desired site and to minimise the concentration of drug in normal tissues (see Senter, P. D. et al (1988) Proc. Natl. Acad. Sci USA 85, 4842-4846; Bagshawe (1987) Br. J. Cancer 56, 531-2; and Bagshawe, K. D. et al (1988) Br. J. Cancer. 58, 700-703.). These are examples of the targeting of drugs in the case of infections, but likewise is applicable to infection. For example, in the case of infection by HIV-1, the early stage of infection is accompanied by the rapid replication of the virus in lymphonodes and is considered to be an event that is crucial to the later progession of the disease. The response of T-cells to the virus and the development of cytolytic T-cells (CTLs) is critically dependent on co-ligation of CD28. Concentrating an inhibitor of GSK-3 at this and other stages of infection will enhance the response of T-cells against virus and the progression of the disease.

A pro-drug approach may also be used without targeting. Accordingly, reference to a GSK3 inhibitor includes reference to a GSK3 inhibitor prodrug.

The invention is now described in more detail by reference to the following, non-limiting, Figures and Examples. Documents referred to herein are hereby incorporated by reference.

### BRIEF DESCRIPTION OF THE DRAWINGS

FIG. 1 shows anti-CD28 ligation induces the phosphorylation of GSK-3α on the inhibitory site Ser-21 in Jurkat T-cells (Panel A, right and left panels) and mouse T-cell hybridoma DC27.10 (Panel B). Detection of phospho-GSKα was conducted by use of anti-phospho-GSKα/β for Ser21/9 in immunoblotting (Cell Signaling, Cat. #9331). Detection of phospho-GSKα was conducted by use of anti-GSKα/β (Biosource International, Cat. #44-610). Panel A, (left panel): lane 1: anti-CD28 plus rabbit anti-mouse (4 μg/ml); lane 2: anti-CD28 (8 μg/ml) plus rabbit anti-mouse; lane 3: rabbit anti-mouse. (upper right panel). Anti-phospho-GSKα/β; (lower right panel) anti-GSKα/β. Lane 1: rabbit anti-mouse; lane 2: anti-CD3 plus rabbit and mouse; lane 3: anti-CD28 plus rabbit and mouse; lane 4: anti-CD3/CD28 plus rabbit anti-mouse. Panel B: (upper panel) Anti-phospho-GSKα/β; (lower panel) anti-GSKα/β. Lane 1: rabbit anti-hamster; lane 2: anti-CD3 plus rabbit anti-hamster; lane 3: anti-CD28 plus rabbit anti-hamster; lane 4: anti-CD3/CD28 plus rabbit anti-hamster.

FIG. 2 shows anti-CD28 ligation induces the phosphorylation of GSK-3β on the inhibitory site Ser-9 in peripheral blood T-cells. The anti-GSK-3 antibodies are the same as used in FIG. 1. Upper panel: Anti-phospho-GSKα/β; lower panel: anti-GSKα/β. Lane 1: rabbit anti-mouse; lane 2: anti-CD3 plus rabbit anti-mouse; lane 3: anti-CD28 plus rabbit anti-mouse; lane 4: anti-CD3 plus anti-CD28 plus rabbit anti-mouse; lane 5: anti-CD28 plus rabbit anti-mouse from cells treated with 100 μM Ly294002; lane 6: anti-CD28 plus rabbit anti-mouse from cells treated with 200 μM Ly294002; lane 7: anti-CD28 plus rabbit anti-mouse from cells treated with 10 μM Cyclosporin A; lane 8: anti-CD28 plus rabbit anti-mouse from cells treated with 20 μM Cyclosporin A; lane 9: anti-CD28 plus rabbit anti-mouse from cells treated with 40 μM Cyclosporin A.

FIG. 3 shows that GSK-3 cooperates with CD28 in the up-regulation of interleukin 2 (IL-2) transcription. Panel A (left panel) Transfection of WT GSK-3 and constitutively active GSK-3 blocked TcR x CD28 induced IL-2 transcription. (right panel) Immunoblot against GSKWT and GSKS94 in cell lysates. Lane 1: mock transfected cells; lane 2: GSKWT transfected cells; lane 3: GSKS94 transfected cells. Panel B: GSK-3 inhibitor SB415286 preferentially cooperates with TcR x CD28 to enhance IL-2 transcription.

FIG. 4 shows that GSK-3 inhibitor SB415286 cooperates to enhance T-cell proliferation in response to the tumor cell line K562.

FIG. 5 shows that GSK-3 inhibitor SB415286 enhances the development of cytolytic T-cells (CTLs) responding to the tumor cell K562 and this function is CD28-dependent. Panel A: GSK-3 inhibitor cooperates to enhance cytolytic T-cell (CTL) killing of K562 cells. Panel B: GSK-3 inhibitor enhancement of cytolytic T-cell (CTL) killing of K562 cells is blocked by the presence of anti-CD28.

FIG. 6 shows that GSK-3 inhibitor Lithium Chloride (LiCl) also enhances cytolytic T-cell (CTL) killing of K562 cells. Cytotoxic activity was also measured and combined with a control where cultures were treated with anti-CD28 mAbs (5 μg/ml). After 6 days, cytolytic T-cells were harvested, washed twice and co-cultured with fresh K562 cells. Cytotoxcity was measured by the CytoTox96 non-radioative cytotoxicity assay from Promega (Southampton, UK).

### DETAILED DESCRIPTION

Reagents

The following reagents are widely available. Nonidet P-40, phenyl methyl sulfonyl fluoride (PMSF) (Sigma, St. Louis, Mo.), sodium dodecyl sulfate (SDS), acrylamide and bisacrylamide (National Diagnostics, Manville, N.J.), Protein A Sepharose beads, Ficoll-Paque (Pharmacia, N.J.), phosphatidyl inositol gamma-32P-ATP (specific activity, 3000 Ci/mmol) (NEN, Mass.). GSK-3 inhibitor SB415286 was purchased commercially from GlaxoSmithKline (London).

Antibodies

Monoclonal antibodies were employed which are specific for CD28: (9.3) (Becton Dickinson, Calif.); and for rabbit anti-mouse antisera (Coulter Immunology, Hialeah, Fla.).

Cells

T lymphoblastoid cell lines, e.g., Jurkat (ATCC TIB 152) were cultured in RPMI-1640 containing 10% (v/v) fetal bovine serum, L-glutamine (2 mM), penicillin (50 U/ml) and streptomycin (50 mg/ml) at 37.degree C and 5% CO.sub.2. Peripheral blood T-cells were isolated from the buffy coat by lymphocyte separation medium (Ficoll-Paque) density gradient centrifugation. The non-adherent cells were cultured in RPMI 1640 supplemented with 10% (v/v) FBS, 100 U/ml penicillin, 100 mg/ml streptomycin and 2 mM L-glutamine. Alternatively, blood was taken from voluntary donors and separated by (Ficoll-Paque) density gradient centrifugation using standard techniques.

Immunoprecipitation

For immunoprecipitations, cells were lysed in ice cold lysis buffer containing 1% TritonX-100 in 20 mM Tris-HCl pH 8.3, 150 mM NaCl. The lysis buffer contained protease and phosphatase inhibitors as described in Prasad et al., 1993, Proc. Natl. Acad. Sci. USA 90:7366. Postnuclear lysates were incubated for 1 hour at 4° C. with the indicated antibodies. Protein A-Sepharose beads (30 ul, Pharmacia), were added and incubated for 1 hour at 4° C. The beads were washed three times in cold lysis buffer and proteins were eluted by boiling for 5 min in SDS sample buffer.

Immunoblotting

Cell lysates (106 cells/lane) or immunoprecipitates were separated by SDS-PAGE and transferred to nitrocellulose for immunoblotting. The membranes were blocked with 5% milk in TBS (10 mM Tris-HCl pH 7.6, 150 mM NaCl) and incubated with the indicated antibody. Bound antibody was revealed with HRP-conjugated rabbit anti-mouse or donkey anti-rabbit antibodies using enhanced chemiluminescence (ECL; Amersham, Arlington Heights, Ill.) or another appropriate secondary antibody, and protein was visualized by enhanced chemiluminescence (ECL, Amersham).

Crosslinking of Surface Receptors

For receptor-crosslinking experiments, peripheral T-cells or Jurkat cells were suspended at a density of 20.times.10.sup.6 cells/ml in RPMI-1640 media containing fetal calf serum (FCS) (2% v/v) and incubated with an anti-CD28 Mab (2-5 ug/mL) or anti-CD3 (2-8 ug/mL) antibody in combination with rabbit anti-mouse (1-2 ug/mL) for various times (usually between 5-1 Smin) at 37 degree C. A sample of rabbit anti-mouse alone served as a negative control.

Measurement of Proliferation

Purified naive T-cells were cultured in 96-well plates at a density of 2×10⁵ cells and activated with anti-CD3/CD28/IgG2α and anti CD3/CD28/CTLA-4 coated beads for 48 hours. After this period of time, cells were washed, and beads were removed by using a magnet. Cells were then restimulated with anti-CD3/CD28 coated beads for further 48 hours. To assay proliferation, cells were pulsed with 1 uCi of [³H] thymidine for the last 18 hours of the indicated periods of time. In parallel, cells were stained for surface GM1 expression as described above.

Measurement of Interleukin 2 (IL-2) Transcription

Jurkat and peripheral T cells were transfected as described (Raab et al. Immunity 15: 921-933, 2001). Briefly, GSTWT and GSKS9A purified cDNAs (10 ug/10⁷ cells) and/or 3×NFAT/AP-1 luciferase reporter (2.5 g/10⁷ cells) from Promega were used. Cells were electroporated using BTX Gene Pulser at 250V, 800 F in 10% FCS, cultured for 15-18 hr, stimulated with OKT3 and/or CD28 and 2 g/ml rabbit anti-mouse at 37° C. for 6 hr, followed by a measurement of luciferase activity using a dual luciferase system kit (Promega, Madison, Wis.) and a luminometer from MicroLumat, (EG&G Berthold, WildBad, Germany). Luciferase units of the experimental vector were normalized to the level of the control vector in each sample.

Induction and Measurement of Cytolytic T-cell Responses to Tumor Cells

Peripheral blood lymphocytes (PBLs) were isolated by Ficoll gradient centrifugation from Buffy coats, or from blood samples (60-120 cc) taken from healthy volunteers. Leukemic cells K562 were first treated with 50 ug/ml Mitomycin C (Sigma) for 1 hour in 5% CO₂ incubator at 37° C. and then washed at least three times with RPMI 1640 to remove the presence of drug. Washed K562 cells and PBLs (1:5 ratio) were then resuspended in RPMI 1640 with 5% fetal calf serum, 1% penicillin-streptomycin and 1% L-glutamine. PBLs and leukemic cells were transferred into 24 well plate. Cultures were either incubated alone, or in the presence of different concentrations of the inhibitor SB415286 (GlaxoSmithKline) or the inhibitor lithium Chloride (1 mM and 5 mM) (Sigma) were added to the cells. Occassionally, PBLs would be preincubated with inhibitor for periods of time (i.e. 5-24 hours) prior to incubation with K562 cells. In either case, cell cultures were then allowed to incubate for 8 days in 5% CO₂ incubator at 37° C. prior to an assessment of cytotoxic function. Over this period, the K562 cells die due to the treatment with mitomycin c.

For an assessment of cytotoxity, effector cells (PBLs) and fresh target cell K562 were added into 96 well plates at various ratios (50:1; 25:1 etc.). Culture medium, effector cells and target cells were added into separate wells as background controls. Cytotoxicity was assessed using a Cytotoxicity Assay kit (Promega) as outlined by the manufacturer.

Results

CD28 Induction of GSK-3 Phosphorylation on Inactivating Sites

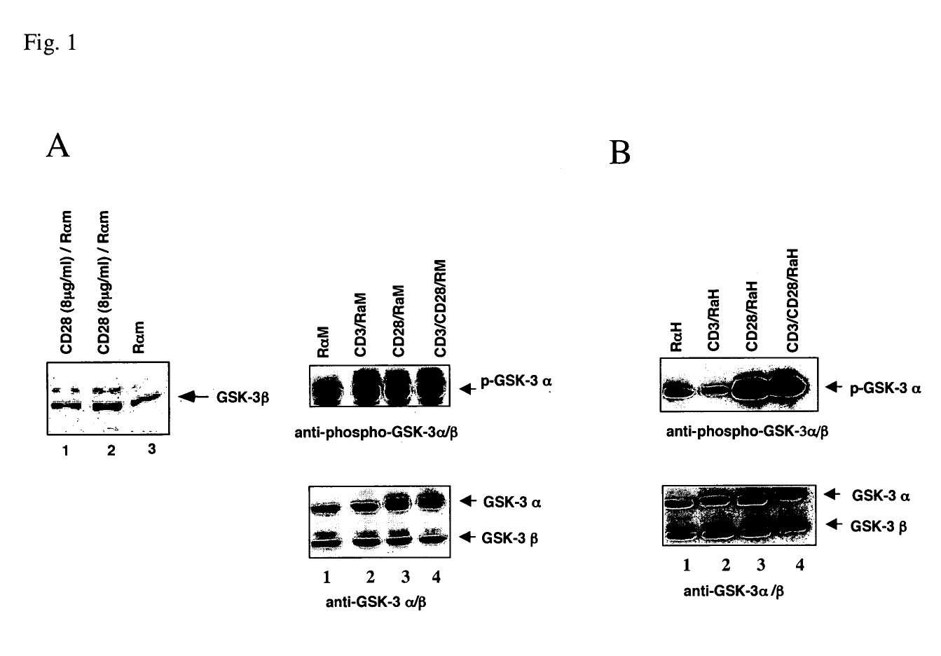

To assess whether CD28 ligation could induce GSK-3 phosphorylation, the T-cell line Jurkat was subjected to anti-CD28 crosslinking followed by immunoblotting with an anti-phospho-specific monoclonal antibody that recognizes phospho-serine 21 on GSK-3α and phospho-serine 9 on GSK-3β (FIG. 1). As shown in FIG. 1A (left panel), anti-CD28 ligation induced an increase in phosphorylation of phospho-serine 9 on GSK-3β and GSK-3α on Ser-21 in Jurkat T-cells (lanes 1,2 vs 3). Further, increasing amounts of the antibody (4 and 8 ug/mL) used in crosslinking increased the level of phosphorylation (lane 1 and 2, respectively). Rabbit anti-mouse served as a negative control (lane 3). These observations indicate that the ligation of CD28 alone can induce the phosphorylation of GSK-3α and β on sites that inhibit the activity of the kinase.

A comparison of GSKα phosphorylation was then made under conditions where anti-CD3, anti-CD28 and a combination of anti-CD3/CD28 antibodies were used to crosslink receptors on the surface of cells (FIG. 1A, right panels). Anti-CD28 ligation induced an increase in phosphorylation of GSK-3α on Ser-21 at levels similar to that induced by anti-CD3 ligation (lane 3 vs 2). Immunoblotting was also conducted using an antibody that recognizes GSK-3 α/β independent of the phosphorylation of the protein (lower panels). This allowed for the identification of the GSK-3β and GSK-3α forms of the kinase.

CD28 ligation also induced GSK-3α phosphorylation on inhibitory sites in a mouse T-cell DC27.10 (FIG. 1B). In this case, anti-CD3 had little if any effect on the phosphorylation status of GSK-3α (lane 2 vs 1). Anti-CD28 induced a marked change in the phosphorylation of GSK-3α on Ser-21 (lane 3 vs 1). The combination of anti-CD3 and CD28 ligation induced levels of phosphorylation similar to that observed with anti-CD28 alone (lane 4 vs. 3). These observations indicate that anti-CD28 crosslinking alone is sufficient to induce marked changes in the phosphorylation of GSK-3 on inhibitory sites. Immunoblotting conducted using an antibody that recognizes GSK-3 α/β independent of the phosphorylation allowed for the identification of the GSK-3α form of the kinase.

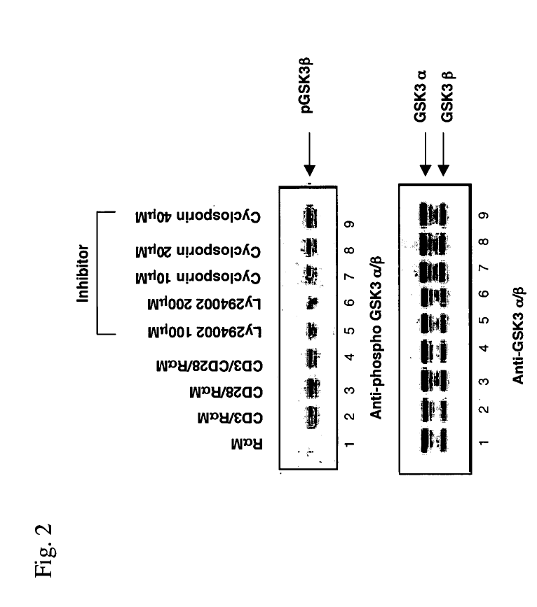

To confirm that CD28 could also induced site specific phosphorylation of GSK-3 in normal primary cells, human peripheral T-cells were subjected to anti-CD28 ligation followed by immunoblotting with an anti-phospho-specific monoclonal antibody that recognizes phospho-serine 21 on GSK-3α and phospho-serine 9 on GSK-3β (FIG. 2, upper panels). In this case, CD28 induced primarily the phosphorylation of GSK-3β on serine 9 (FIG. 2, lane 3 vs 1). In this case, the level of phosphorylation in control cells was negligible (lane 1), while anti-CD28 ligation alone induced greater phosphorylation than observed for anti-CD3 crosslinking (lane 2 vs 3). Overall, our data shown that these data clearly demonstrate that CD28 ligation can each induce the phosphorylation of the kinase GSK-3β on Ser 9 in human peripheral T-cells.

Two distinct CD28 signaling pathways have been described, one dependent on calcium and cyclosporin sensitive and another one cyclosporine A (CsA) insensitive (Ghosh et al. Blood 99: 4517-4524, 2002). In an attempt to assess whether PI 3-kinase and/or the calcinuerin-cyclophilin pathway operates upstream of the CD28-GSK-3 pathway, cells were exposed to the by PI 3-kinase inhibitor, wortmannin and the calcinuerin-cyclophilin inhibitor Cyclosporin A. Treatment of peripheral T-cells with wortmannin at 100 and 200 nM for 30 min partially decreased CD28 induced phosphorylation of GSK-3 on Ser-9 (FIG. 2, lanes 5,6). By contrast, cells were treated for 30 min with various concentrations of cyclosporin A and then stimulated with anti-CD28 mAbs showed the same level of phosphorylation as found in the untreated samples. GSK-3 β phosphorylation was insensitive to cyclosporin A, even at a concentration of 40 _M cyclosporine (lanes 7-9). As a control for GSK-3 expression, immunoblotting of lysates showed equal amounts of GSK-3 in the different cells (lower panel). These data show that CD28 inactivation of GSK3β depends on PI 3K activity, but not on the TcR-driven calcinuerin-cyclophilin pathway. This further underlines the independence of the TcR and CD28 mediated pathways.

CD28 Cooperation with SB415286 in the Potentiation of GSK-3 Inactivation

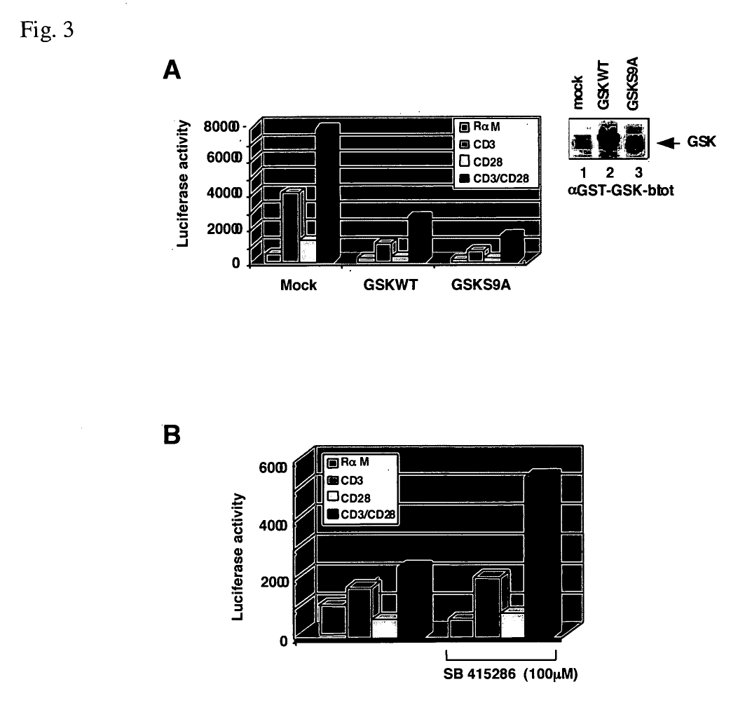

The ability of CD28 to regulate GSK-3 might have an effect on the enhancing effect of CD28 on TcR driven interleukin 2 (IL-2) transcription. To address this, DC27.10 cells that had been stably transfected with human CD28 were transiently transfected with either wild-type, or constitutively active GSK-3 (GSK-3S9A) in the presence of a NFAT-AP-1-luciferase based IL-2 reporter plasmid. Transcriptional activity assessed upon stimulation with anti-CD3, anti-CD28 and anti-CD3/CD28 mAbs (FIG. 3A). Under these conditions, anti-CD3 induced IL-2 transcription, which in turn was greatly potentiated by anti-CD28 co-ligation. By contrast, over-expression of GSK-3WT and GSK-3S9A significantly reduced both TcR/CD3 and TcR/CD3-CD28 mediated IL-2 transcription when compared to mock transfected cells. Importantly, the expression of wild-type, or constitutively active GSK-3 (GSK-3S9A) also inhibited the potentiating effect of anti-CD28 alone on activation. In the presence of GSKS9A, anti-CD3/CD28 co-crosslinking induced levels of IL-2 transcription that were even below the level observed with TcR/CD3 ligation in mock transfected cells and 7-8 times lower than that observed with combined ligation of TcR and CD28 on mock transfected cells. As a control for expression, anti-GST showed the presence of expressed GSKWT and GSKS9A (upper right panel). Overall, these data confirm by a different approach that CD28 and TcR x CD28 co-stimulation employs the GSK pathway in its amplification of IL-2 transcription.

To further assess whether the CD28 co-stimulation is connected to GSK-3, T-cells were activated by anti-CD3, or anti-CD3/CD28, in the presence or absence of 100 uM GSK-3 inhibitor SB415286. As above, an assessment of IL-2 gene activation was determined using cells that had been transfected with a NF-AT/AP-1 IL-2 promoter construct. Under these conditions, anti-CD3 activated transcription was slightly increased by co-ligation with anti-CD28. However, the addition of SB415286 preferentially increased the response in the context of CD28 co-ligation (FIG. 3B). A slight effect was observed on anti-CD3 induced gene activation; however, this was some 10-fold lower than that observed in the context of anti-CD28 co-ligation. Overall, these finding demonstrate a preferential connection between CD28 co-ligation and the ability of inactivators of GSK-3 to potentiate T-cell cytokine responses.

SB415286 Enhances Cytolytic T-Cell Responses Against Tumor Cell Line K562 in a CD28 Dependent Manner

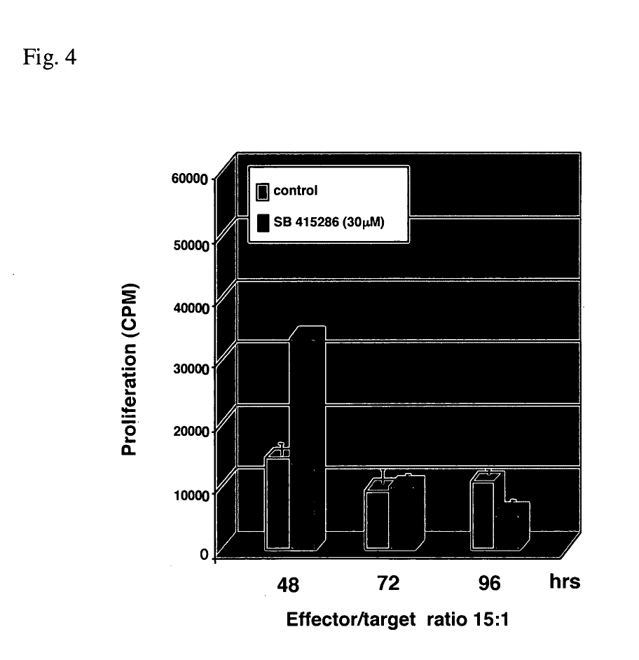

Previous studies have documented a role of CD28 is enhancing proliferation and the generation of a cytotoxic T-cell response to tumor cells (Chen et al Cell 1992 71:1093-102). These anti-tumor CTL responses against both melanoma and carcinoma tumours are crucially dependent on CD28 mediated co-signaling (Haynes J Immunol 2002 169:5780-6; Chen et al Cell 1992 71:1093-102; Hu et al J Immunol 2002 169:4897-904). Given the ability of CD28 to inactivate GSK-3, it was next assessed whether the inactivation of GSK-3 could play the crucial role in potentiating these T-cell responses to tumor cells. Peripheral T-cells were exposed to the BcR-Abl induced tumor erythroleukemia cell line K562 (that had been treated with mitomycin C) and assessed for proliferation and the generation of a CTL response (FIG. 4). T-cells were found to undergo proliferation from 48-96 hours as measured by 3H-thymidine incorporation. Under these conditions, the response of T-cells was greatly enhanced by exposure to SB415286. Treatment with SB415286 increased the level of response by two-three fold at 48 hours. These data clearly show that inhibition of GSK-3 can potentiate the proliferative response to tumor cells.

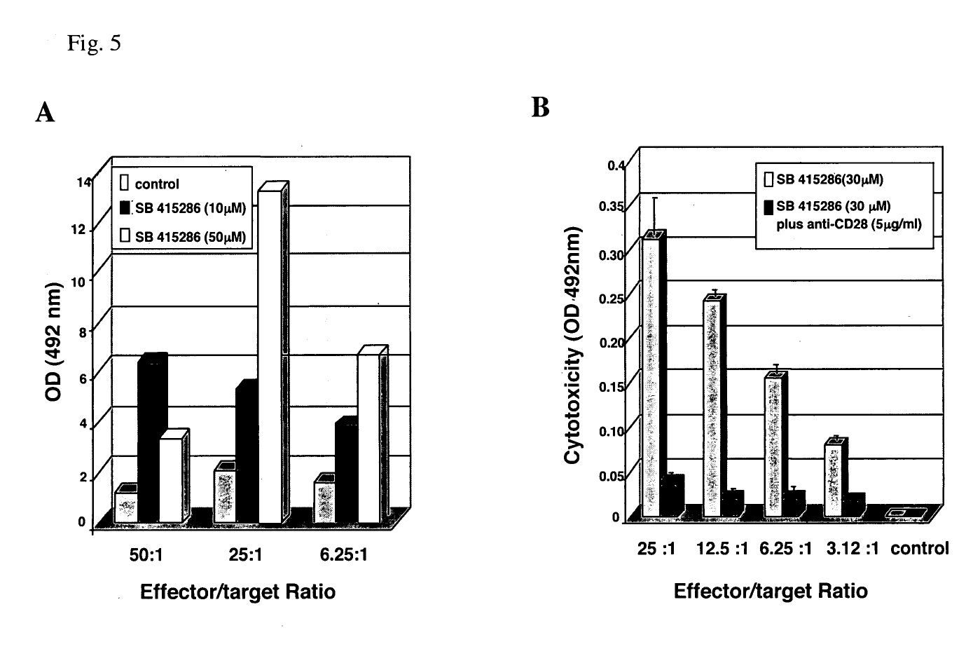

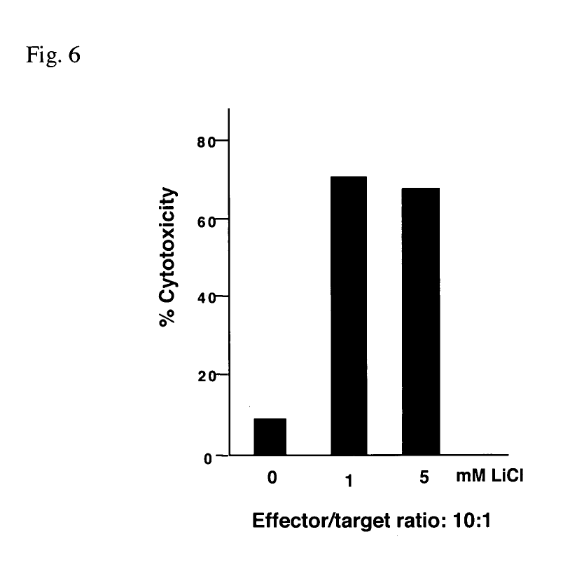

In addition to proliferation, SB415286 enhanced the development of a CTLs response against the K562 cells (FIG. 5). Peripheral T-cells were co-cultured with K562 cells in the absence or presence of the GSK-3 inhibitor SB415286 for 6 days followed by a measurement of cytolytic T-cell function. Cytotoxicity was measured by CytoTox96® assay. Under these conditions, the inhibitor had a major effect in potentiating cytolytic T-cell response (FIG. 5A). The response was potentiated with 10 um by some 5-6-fold in response at a 50:1 effector target ratio and at 50 um at a 25:1 ratio. Further, the CD28-dependency of the response was shown by the fact that anti-CD28 blocked the cytotoxicity at all T-cell/APC ratios (FIG. 5B). This dependency on CD28 is consistent with the ability of anti-CD28 to induce GSK-3 phosphorylation on Ser-9 (FIG. 1) and the ability of SB415286 to cooperate more effectively with anti-CD3/CD28 co-ligation than anti-CD3 alone (FIG. 3B). It also demonstrates that the potentiation of cytolytic activity is not simply due to the clonal expansion of reactive T-cells (which will also occur) since the cytolytic assay was normalised on a per cell basis. Lastly, consistent with an effect mediated via GSK-3, another inhibitor of GSK-3 also potentiated CTL killing of targets (FIG. 6).

### EXAMPLE 1

The invention may be used to regenerate and enhance an immune response involving the production of cytokines (interleukin 2,4, interferony etc), the development of cytotoxic T-cells, and the reconstitution of immune cells in an immunocompromised individual with immunodeficiency due to chemotherapy or as caused by an infectious agent scuh as the Human Immunodeficiency Virus (HIV-1). Peripheral blood lymphocytes and/or purified T-cells purified from human blood may be exposed in vitro to various concentrations of a drug that inhibits GSK-3a/b, either in the absence or presence of reagents that ligate CD28 such as anti-CD28, or CD80/86. Cells are left for various times at 37° C. or RT, and re-infused into the patient at various cell concentrations.

### EXAMPLE 2

The invention may be used to enhance the development of an immune response and the generation of a cytotoxic T-cell response (CTL) in the treatment of patients with various infections caused by virus', bacteria, fungi or prions. Peripheral blood lymphocytes and/or purified T-cells purified from human blood may be exposed in vitro to various concentrations of a drug that inhibits GSK-3a/b, either in the absence or presence of reagents that ligate CD28 such as anti-CD28, or CD80/86. Cells are left for various times at 37° C. or RT, and re-infused into the patient at various cell concentrations. Alternatively, exposure to the inhibitor would be combined with the presentation of specific antigens associated with the virus or bacteria, alone or in conjunction with dendritic cell vaccines (ie dendritic cells displaying specific antigens). Dendritic cells could also be infected with the appropriate virus and used as a vaccine.

### EXAMPLE 3

The invention may be used to boost the development of an immune response and the generation of a cytotoxic T-cell response (CTL) in the treatment of patients with a defect in the function of the CD28 co-receptor. Peripheral blood lymphocytes and/or purified T-cells purified from human blood, exposed in vitro to various concentrations of a drug that inhibits GSK-3a/b. Cells are left for various times at 37° C. or RT, and re-infused into the patient at various cell concentrations.

### EXAMPLE 4

The invention may be used to reverse the development of non-responsiveness or anergy in the context of infection. Peripheral blood lymphocytes and/or purified T-cells purified from human blood may be exposed in vitro to various concentrations of a drug that inhibits GSK-3a/b, either in the absence or presence of reagents that ligate CD28 such as anti-CD28, or CD80/86. Cells are left for various times at 37° C. or RT, and re-infused into the patient at various cell concentrations.

### EXAMPLE 5

The invention provides the development of transgenic nonhuman animals that comprise transgenes that alter CD28 inactivation of GSK-3β or β and their use in investigating infection. The invention also provides methods for the identification of novel drugs or peptides useful in treating infections that interfere or augment the CD28 inactivation of the GSK-3α,β pathway. Also within the invention is the use of isolated nucleic acid sequences that encode the peptides or proteins that regulate the CD28-GSK-3 pathway. For example, the over-expression of a protein will interfere with CD28 function, or gene knock-out technology is used to delete a gene that affects CD28 function. Alternatively, gene knock-out technology is used to delete a gene that affects TcR signaling, thereby necessitating the engagement of CD28 to provide signals needed for the response of the T-cell to antigen, or a foreign graft. Using gene therapy techniques, DNA encoding such proteins and peptides is taken up by cells and expressed in the cytoplasm. The DNA may be introduced into target cells in the bloodstream or other tissues of the patient by standard vectors and/or gene delivery systems. Suitable gene delivery systems may include liposomes, receptor-mediated delivery systems, naked DNA, and viral vectors such as herpes viruses, retroviruses, and adenoviruses, among others.

### EXAMPLE 6

The invention provides screens for therapeutically useful modulators for treating infections by a virus, bacterium, fungi or prions. A screening method for identifying compounds useful in treating infections by identifying compounds capable of modulating CD28 inactivation of GSK-3 can be carried out. The assay utilizes a cell that expresses CD28 and GSK-3. The cell is most preferably a T cell such as HPB-ALL or Jurkat, but may be any type of cell which expresses CD28 on its surface and GSK-3 in its cytoplasm, e.g., a cell transfected with cDNAs encoding CD28 and/or GSK-3. A sample of cells is incubated in the presence or in the absence of a candidate compound. A reference point could be established under standard conditions and the results from any assay compared to the pre-established standard as the control. Alternatively, controls could be run in parallel with each screening assay. Cell surface CD28 is cross-linked with, e.g., a CD28-specific antibody or a CD28 ligand, such as CD80/86. Cell lysates would be subject to immunoblotting using a phospho-specific GSK-3 antibody to Ser-9, or the equivalent residues on other GSK-3 family members. Also, GSK-3 is precipitated from a cell lysate and by immunoblotting with an anti-GSK-3 antibody specific for phospho specific forms of GSK-3. The intensity of staining can be quantitated by means of standard densitometric techniques.

A method which measures the substitution of CD28 mediated function by inhibiting of GSK-3 by a given compound can also be used to identify compounds capable of modulating T cell activation and useful in treating infections. Using cells which express CD28 and GSK-3, cell surface CD28 is crosslinked with anti-CD28 or forms of CD80 or CD86 and followed by immunoblotting with an anti-GSK-3 antibody specific for phospho specific forms of GSK-3. Also, GSK-3 is precipitated from a cell lysate and by immunoblotting with an anti-GSK-3 antibody specific for phospho specific forms of GSK-3.

### EXAMPLE 7

Treatment as indicated in the preceding Examples may also be carried out by administering to the patient a therapeutically effective amounts of an inhibitor of GSK-3.beta (or alpha), either alone as a CD28-substitute signal, or in conjunction with reagents (i.e. anti-CD28/CD80/CD86) that ligate CD28.

### EXAMPLE 8

The invention provides a method for expanding lymphoid cells that could be grown from organs, for example tissue-infiltrating lymphocytes. Organs include lymph-nodes, liver, bone marrow, lung, the pancreas and others. The cells are administered an inhibitor of GSK-3.beta (or alpha), either alone as a CD28-substitute signal, or in conjunction with reagents (i.e. anti-CD28/CD80/CD86) that ligate CD28. This could be done together with the presentation of specific peptide antigens in vitro and the treated cells may be administered to patients.

### OTHER REFERENCES

Aruffo et al. Molecular cloning of a CD28 by a high-efficiency COS cell expression system. Proc. Natl. Acad. Sci USA 84: 8573-8577, 1987.
Avni et al. T(H) cell differentiation is accompanied by dynamic changes in histone acetylation of cytokine genes. Nat. Immunol. 3: 643-651, 2002.
Burr et al. Cutting edge: distinct motifs within CD28 regulate cell proliferation and induction of Bcl-XL. J. Immunol. 166: 5331-5335, 2001.
Cai et al. Selective CD28pYMNM mutations implicate phosphatidylinositol 3-kinase in CD86-CD28-mediated costimulation. Immunity 3: 417-426, 1995.
Cantrell, Protein kinase B (Akt) regulation and function in T lymphocytes. Seminars in Immunology 14: 19-26, 2002.
Del Rio et al. Characterization of the effects of lithium and inositol on phoshoinositide turnover in cerebellar granule cells in primary culture. J. Neurochem 66: 517-524, 1996.
Wang et al., Lithium inhibition of PKC activation and activation-induced serotonin release. Psychopharmacology 99: 213-218, 1989.
Frame et al. GSK3 takes centre stage more than 20 years after its discovery. Biochem. J. 359: 1-16, 2001
Frauwirth et al. The CD28 signaling pathway regulates glucose metabolism. Immunity 16: 769-777, 2002.
Fruman et al. Impaired B cell development and proliferation in the absence of phosphoinositide 3-kinase p85 alpha. Science 283: 393-397,1999.
Ghosh et al. Effect of rapamycin on the cyclosporin Aresistant CD28-mediated costimulatory pathway. Blood 99: 4517-4524, 2002.
Harada et al. Critical requirement for the membrane-proximal cytosolic tyrosine residue for CD28-mediated costimulation in vivo. J. Immunol. 166: 3797-3803, 2001.
Hoeflich, et al. Requirement for glycogen synthase kinase-3.beta. in cell survival and NF-kB activation. Nature, 406. (Jul. 6, 2000), pp 86-90.
Holdorf et al. Proline residues in CD28 and Src homolgy (SH)3 domain of LCK are required for T cell costimulation. J. Exp. Med. 190: 375-384, 1999.
Iwakami et al. Replication-deficient adenovirus-mediated transfer of B7-1 (CD80) cDNA induces anti-tumour immunity in isolated human lung cancer. Respirology 6: 135-144, 2001.
Kane et al. Akt provides the CD28 costimulatory signal for up-regulation of IL-2 and IFN-gamma but not TH2 cytokines. Nat. Immunol. 2: 37-44, 2001.
Kim et al. Growth factor receptor-bound protein 2 SH2/SH3 domain binding to CD28 and its role in co-signaling. J. Biol. Chem. 273: 296-301, 1998.
Linsley et al. The Role of the CD28 Receptor During T Cell Responses to Antigen. Annu. Rev. Immunol. 11:191-212(1993).
Lu et al. CD28-Induced T Cell Activation-Evidence for a Protein-Tyrosine Kinase Signal Transduction Pathway. J. Immunol.149: 24-29 (1992).
Murphy et al. Endothelial cells stimulate T cell NFAT nuclear translocation i the presence of cyclosporin A: involvement of the wnt/glycogen synthase kinase-3 beta pathway. J. Immunol. 169: 3717-3725, 2002.
Ohteki et al. Negative regulation of T cell proliferation and interleukin-2 production by the serine threonine kinase GSK-3. J. Exp. Med. 192: 99-104, 2000.
Okkenhaug et al. A point mutation in CD28 distinguishes proliferative signals from survival signals. Nat Immunol 325: 325-332, 2001.
Pages et al. Binding of phosphatidylinositol-3-OH kinase to CD28 is required for T-cell signaling. Nature 369: 327-329,1994.
Prasad et al. Src-homology 3 domain of protein kinase p. 59.sup.fyn mediates binding to phosphatidylinositol 3-kinase in T cells. Proc. Natl. Acad. Sci. USA 90:7366-7370 (1993).
Prasad et al. T-cell antigen CD28 interacts with the lipid kinase phosphatidylinositol 3-kinase by a cytoplasmic Tyr(P)-Met-Xaa-Met motif. Proc. Natl. Acad. Sci. USA 91: 2834-2838, 1994.
Raab et al. CD28 signaling via Vav/SLP-76 adaptors: regulation of cytokine transcription independent of TCR ligation. Immunity 15: 921-933, 2001.
Rudd. Upstream-downstream: CD28 cosignaling pathways and T cell function. Immunity 4: 1-20, 1996.
Schwartz et al. A cell culture model for T lymphocyte clonal anergy. Science 248: 1349-1356, 1990.
Truitt et al. Stimulation of CD28 triggers an association between CD28 and phosphatidylinositol-3-kinase in Jurkat cells J. Exp. Med. 179: 1071-1076, 1994.
Wang et al. Regulation of TRAIL expression by the phosphatidylinositol 3-kinase/Akt/GSK-3 pathway in human colon cancer cells. J. Biol. Chem. 277: 36602-36610, 2002.
Zhen et al. Lithium regulates protein tyrosine phosphatase activity in vitro and in vivo. Psychopharmacology 162: 379-384, 2002.

1. A method of enhancing CD28 mediated and dependent T-cell responses against viral, bacterial, fungal or prion infections in a mammalian subject comprising administering a drug to said subject, wherein said drug enhances or prolongs GSK-3α or β inactivation.

2. A medicament for enhancing CD28 mediated and dependent T-cell responses against infections in a mammalian subject, wherein the medicament comprises a drug which enhances or prolongs GSK-3α or β inactivation.

3. A method of enhancing CD28 mediated and dependent T-cell responses against infections comprising administering a drug to isolated peripheral T-cells or blood of a mammalian subject, wherein said drug enhances or prolongs GSK-3α or β inactivation, for example CD28-mediated GSK-3α or β inactivation.

4. A medicament for enhancing CD28 mediated and dependent T-cell responses against infections in a mammalian subject, wherein the medicament comprises a drug and is for administering to isolated peripheral T-cells or blood of said subject, wherein said drug enhances or prolongs GSK-3α or β inactivation, for example CD28-mediated GSK-3α or β inactivation.

5. The method of claim 1 or 3, wherein the drug is a peptide.

6. The medicament of claim 2 or 4 wherein the drug is a peptide.

7. The method of claim 1 or 3, wherein the said drug is SB415286 from GlaxoSmithKline, or a related GSK-3 inhibitory compound such as a 3-indolyl-4-phenyl-1H-pyrrole-2,5-dione derivative or novel pyridine or pyrimidine derivative from another company, or wherein the said drug is lithium chloride or a related compound.

8. The medicament of claim 2 or 4, wherein the said drug is SB415286 from GlaxoSmithKline, or a related GSK-3 inhibitory compound such as a 3-indolyl-4-phenyl-1H-pyrrole-Z5-dione derivative or novel pyridine or pyrimidine derivative from another company, or wherein the said drug is lithium chloride or a related compound.

9. The method of claim 1 or claim 3, wherein the drug can provide a CD28-substitute signal, or act in conjunction with reagents (i.e. anti-CD28; CD80/CD86) that ligate CD28.

10. The method of claim 1 or claim 3 leading to enhanced cytokine production.

11. The method of claim 1 or claim 3 leading to increased T-cell proliferation and clonal expansion.

12. The method of claim 1 or claim 3 leading to increased cytotoxic T-cell expansion and function.

13. A method for screening or identifying a compound useful in treating infections, comprising identifying inhibitory and stimulatory compounds capable of modulating CD28 inactivation of GSK-3.

14. The method according to claim 1 or claim 3, futher comprising administering an agent to said subject, wherein said agent is capable of ligating CD28 to peripheral blood lymphocytes and/or T-cell in said subject.

15. The method according to claim 1 or claim 3 for expanding lymphoid cells that could be grown from tissues infected with a pathogen such as a virus, bacterial, fungi or prion, otherwise known as tissue-infiltrating lymphocytes.

16. A medicament comprising a reagent (for example anti-CD28; CD80/CD86) that ligates CD28 for enhancing CD28 mediated and dependent T-cell responses against infections in a mammalian subject, wherein the subject is a subject administered a non-peptide drug (either directly or via administration of the drug to isolated peripheral T-cells or blood of said subject) which enhances or prolongs GSK-3α or β inactivation, for example CD28-mediated GSK-3α or β inactivation.

17. A medicament comprising a reagent (for example anti-CD28; CD80/CD86) that ligates CD28 for enhancing CD28 mediated and dependent T-cell responses. against infections in a mammalian subject, wherein the medicament is for administering to isolated peripheral T-cells or blood of said subject and wherein a drug which enhances or prolongs GSK-3α or β inactivation, for example CD28-mediated GSK-3α or β inactivation is administered to the isolated peripheral T-cells or blood of said subject or directly to the subject.

18. A medicament for treating infections, for example a infection as indicated above, comprising a composition or kit of parts comprising a GSK3 inhibitor and a reagent that ligates CD28.
